(19) United States
(12) Patent Application Publication
(10) Pub. No.: US 2011/0223215 A1
(43) Pub. Date: Sep. 15, 2011

(54) PROCESS FOR MAKING A TOPICAL SCRUB

(75) Inventor: Thomas G. Mason, Los Angeles, CA (US)

(73) Assignee: The Regents of the University of California, Oakland, CA (US)

(21) Appl. No.: 13/129,550

(22) PCT Filed: Dec. 2, 2009
(86) PCT No.: PCT/US2009/066428

§ 371 Date: May 16, 2011

Related U.S. Application Data
(60) Provisional application No. 61/193,469, filed on Dec. 2, 2008.

(51) Int. Cl. A61K 9/00; A61K 9/10; A61K 9/107

(52) U.S. Cl. 424/400

(57) ABSTRACT

A method of producing a topical scrub includes providing a particle material suitable for producing a customized anisotropic particle; producing a plurality of customized anisotropic particles composed of the particle material, each customized anisotropic particle having a particle shape, a particle volume, and a particle composition; and dispersing the plurality of customized anisotropic particles in a fluid material to form a dispersion of customized anisotropic particles in the fluid material. At least a feature of the particle shape facilitates embedding at least a portion of the plurality of customized anisotropic particles into a topical surface by a scrubbing action.

### CROSS-REFERENCE TO RELATED APPLICATION

This application claims priority to U.S. Provisional Application No. 61/193,469 filed Dec. 2, 2008, the entire contents of which is hereby incorporated by reference.

### BACKGROUND

1. Field of Invention

This application relates to processes, compositions, and systems for making a topical scrub containing particle structures dispersed in a fluid, and more particularly processes, compositions, and systems for making a topical scrub containing particle structures dispersed in a fluid using customized anisotropic particles containing biocompatible and/or bioactive materials.

2. Discussion of Related Art

The contents of all references, including articles, published patent applications and patents referred to anywhere in this specification are hereby incorporated by reference.

Lithographic methods (Madou, M. J. Fundamentals of microfabrication: The science of miniaturization. 2nd ed.; CRC Press: Boca Raton, 2002) can be used to design and mass-produce large numbers of custom-shaped particles dispersed in a fluid (Hernandez, C. J.; Mason, T. G. Colloidal alphabet soup: Monodisperse dispersions of shape-designed LithoParticles. J. Phys. Chem. C 2007, 111, 4477-4480; Hernandez, C. J.; Zhao, K.; Mason, T. G. Pillar-deposition particle templating: A high-throughput synthetic route for producing LithoParticles. Soft Materials 2007, 5, 1-11; Hernandez, C. J.; Zhao, K.; Mason, T. G. Well-deposition particle templating: Rapid mass-production of LithoParticles without mechanical imprinting. Soft Materials 2007, 5, 13-31; Higurashi, E.; Ukita, H.; Tanaka, H.; Ohguchi, O. Optically induced rotation of anisotropic micro-objects fabricated by surface micromachining. Appl. Phys. Lett. 1994, 64, 2209-2210; Rolland, J. P.; Maynor, B. W.; Euliss, L. E.; Exner, A. E.; Denison, G. M.; DeSimone, J. M. Direct fabrication of monodisperse shape-specific nanobiomaterials through imprinting. J. Am. Chem. Soc. 2005, 127, 10096-10100; Brown, A. B. D.; Smith, C. G.; Rennie, A. R. Fabricating colloidal particles with photolithography and their interactions at an air-water interface. Phys. Rev. E 2000, 62, 951-960; Sullivan, M.; Zhao, K.; Harrison, C.; Austin, R. H.; Megens, M.; Hollingsworth, A.; Russel, W. B.; Cheng, Z.; Mason, T. G.; Chaikin, P. M. Control of colloids with gravity, temperature gradients, and electric fields. J. Phys. Condens. Matter 2003, 15, S11-S18). In addition to top-down lithographic methods that facilitate the direct design of millimeter and sub-millimeter particles that have customized shapes and sizes, non-spherical particles can also be made and sometimes even designed by bottom-up processes, such as growth of colloidal particles (e.g. nanorods) in solution. Regardless of the methods of production, having a means of designing the shapes and sizes of the particles is potentially useful for creating desired particle shapes and sizes that may have enhanced functionality owing to pre-specified geometrical features inherent in their shapes and sizes.

Among methods of producing shape-designed particles which typically have sub-millimeter maximum spatial dimensions, the methods of spatially patterned radiation (U.S. Pat. App. Ser. No. 12/377,773 which claims priority to PCT/US07/18365 and 60/838,160 all of which are incorporated herein by reference) and the method of relief deposition templating (U.S. Pat. App. Ser. No. 12/563,907 which claims priority to PCT/US08/003679, 61/100,471 and 60/918,896 all of which are incorporated herein by reference) are among the best suited for mass-producing such particles. In particular, the method of relief deposition templating can provide large numbers of particles without a need for repeated lithographic exposure after the template has been produced. Other methods for producing shape-designed particles are relief radiation templating (U.S. Pat. App. Ser. No. 12/575,920 which claims priority to 61/103,777 both of which are incorporated herein by reference) and two-patterned surface imprinting (U.S. Pat. App. Ser. No. 12/579,226 which claims priority to 61/105,232 both of which are incorporated herein by reference).

For the purposes of this specification, the word “topical” typically refers to surfaces of a body of an organism that is living or at least was once alive, including but not limited to humans, mammals (e.g. dogs, cats, horses, primates, cattle, and sheep), fish, birds, reptiles, amphibians, insects, arachnids, plants, fungi, and trees. For example, in the case of humans, such surfaces of a body typically include but are not limited to: skin, lips, nails, cuticles, hair, follicles, membranes, ocular surfaces, oral surfaces, tongue surfaces, surfaces of gums and teeth, nasal surfaces, ear surfaces, anal surfaces, urethral surfaces, surfaces of mucous membranes, surfaces of open wounds, surfaces of internal organs that have been exposed by surgery, and surfaces of reproductive organs (e.g. vagina, cervix, labia, penis, and scrotum).

One common problem that frequently arises during the use of topical agents, such as creams, lotions, ointments, emulsions, gels, sprays, and a wide variety of topical prescription drugs, over-the-counter drugs, and personal care products, is that these topical agents must be applied repeatedly over a sustained period of time to an affected area in order to cure the cause of the symptoms effectively. Frequently, the user of these products, after some initial improvement and possibly a disappearance of most of the symptoms, does not follow the directions for repeated topical application over the entire recommended duration of treatment, and this sometimes leads to a recurrence of the undesired symptoms, possibly caused by the reproduction of harmful microbes that have an even greater resistance to treatment. An example of this is simple athlete's foot or jock itch, generally caused by a fungal infection; application of a product is often recommended over about two weeks, yet many users of anti-fungal products that could cure these ailments do not apply the topical agent over this entire recommended period of time. Thus, one of the key problems in the administration of topical agents that have therapeutic properties is failure to complete the full course of recommended medication through repeated applications over a sustained duration. Such repeated applications of topical agents, if followed, would have delivered a desired dose of a therapeutic agent (e.g. anti-fungal drug molecules) over a necessary sustained duration required for full treatment.

It would be therefore highly advantageous to design and create a topical material that could provide a desired dose over a sustained duration after only one topical application of that material, whether by the person who has the condition (e.g. self-treatment) or by a health-care professional (e.g. a doctor or a nurse). Since technologies now exist for designing and mass-producing custom-shaped particles, it would be desirable to create and incorporate custom-shaped and custom-sized particles into a topical agent, such as a topical scrub. Although topical scrubs exist (e.g. such as those that through repeated application are used for their abrasive properties to treat poison ivy), past approaches in the general area of topical scrubs have typically focused on including particles that abrade the skin. These past approaches to topical scrubs have lacked the combination of custom-designing the compositions, shapes, and sizes of particles to facilitate embedding of the particles into topical surfaces and to deliver a desired profile of time release of a therapeutic (e.g. bio-active) agent over a sustained period of time. It is precisely this unique combination of features in a topical material that would provide a highly useful material composition and enable a process for one-time topical application of a topical scrub containing custom-shaped and custom-sized anisotropic particles that are typically bio-compatible and bio-active.

For the purposes of this specification, we refer to a particle having a customized shape, customized size, and/or customized composition as a customized anisotropic particle. In some of the above-noted references, the lithographic particles or LithoParticles can provide examples of customized anisotropic particles according to some embodiments of the current invention.

### SUMMARY

A method of producing a topical scrub according to an embodiment of the current invention includes providing a particle material suitable for producing a customized anisotropic particle; producing a plurality of customized anisotropic particles composed of the particle material, each customized anisotropic particle having a particle shape, a particle volume, and a particle composition; and dispersing the plurality of customized anisotropic particles in a fluid material to form a dispersion of customized anisotropic particles in the fluid material. At least a feature of the particle shape facilitates embedding at least a portion of the plurality of customized anisotropic particles into a topical surface by a scrubbing action.

A multi-component composition according to an embodiment of the current invention has a first material component, in which customized anisotropic particles can be dispersed, and a plurality of customized anisotropic particles dispersed in the first material component, each customized anisotropic particle having a particle shape, a particle volume, and a particle composition. At least a feature of the particle shape facilitates embedding at least a portion of the plurality of customized anisotropic particles into a topical surface by a scrubbing action, and the plurality of customized anisotropic particles is at least 100 particles.

A system for producing a topical scrub according to an embodiment of the current invention has a particle production system structured to produce a plurality of customized anisotropic particles and a dispersion system arranged proximate the particle production system to be able to receive a plurality of the customized anisotropic particles and to be able to receive a fluid material, and through which the plurality of customized anisotropic particles can be dispersed in the fluid material. Each of the plurality of customized anisotropic particles has at least a feature of a shape that facilitates embedding of at least a portion of the customized anisotropic particles into a topical surface by a scrubbing action.

### BRIEF DESCRIPTION OF THE DRAWINGS

The invention is better understood by reading the following detailed description with reference to the accompanying figures in which:

FIG. 1(a) is a schematic illustration of a portion of a pillar template for producing customized anisotropic particles suitable for a topical scrub, according to an embodiment of the current invention. According to an embodiment of the current invention, the pillars shown have a triangular cross-section at their upper surface portion suitable for producing customized anisotropic particles that have pointed triangular features through the process of pillar deposition templating, a form of relief deposition templating. Typically, lateral cross-sectional dimensions of the pillars on the template are sub-millimeter, and the height of the tops of the pillars above the lower surface portion of the template is typically many times larger than the largest dimension of the particles to be produced. Optionally, the pillars on the pillar template can be coated with a release material to facilitate the release (i.e. separation) of desired particles from the pillar template.

FIG. 1(b) is a schematic illustration of a portion of a pillar template (shown in FIG. 1(a)) that has been coated with a particle material (shown in dark shading) suitable for producing customized anisotropic particles to be incorporated into a topical scrub, according to an embodiment of the current invention. According to an embodiment of the current invention, the upper surface portions (i.e. tops) of the pillars have been coated with a particle material and this material has been solidified to produce a plurality of discrete customized anisotropic particles (shown as dark triangular prisms) suitable for being incorporated into a topical scrub.

FIG. 1(c) is a schematic illustration of a plurality of customized anisotropic particles (shown as triangular prisms) composed of a particle material suitable for a topical scrub that have been produced, subsequent to being released from a pillar template (shown in FIG. 1(b)), using the process of pillar deposition templating, according to an embodiment of the current invention. As is typical of the release step in pillar deposition templating, the particles have been released into a fluid material (shown in light shading). Optionally, a stabilizing material can be present in the fluid material to inhibit aggregation or agglomeration of particles subsequent to release from the pillar template.

FIG. 1(d) is a schematic illustration of a dispersion of a plurality of customized anisotropic particles obtained from a particle production process (e.g. such as a process shown in FIGS. 1(a), 1(b), and 1(c)) that have been incorporated into a gel-like fluid material, wherein said particles have a composition of a particle material that is suitable for a topical scrub, according to an embodiment of the current invention. According to an embodiment of the current invention, the fluid material can be made gel-like (i.e. viscoelastic) by adding a polymer material (shown as lines) to the fluid material containing the custom-shaped and custom-sized particles, wherein said polymer material forms an entanglement network, a physical network, a chemical network, a cross-linked network, or a combination thereof. Such polymer materials that function as rheological modifiers are typically referred to as thickening agents. According to another embodiment of the current invention, an average mesh size and/or an average entanglement length characterizing a solution of a polymer material in a fluid material (e.g. defined in de Gennes, P. G., Scaling Concepts in Polymer Physics. Cornell University Press (1979)) is typically about the same as or smaller than the maximum dimension of a particle.

FIG. 1(e) is a schematic illustration of a dispersion of a plurality of customized anisotropic particles (dark triangular prisms) that have customized shapes and sizes and that are composed of a particle material suitable for a topical scrub. For application to a topical surface, a topical scrub containing a plurality of customized anisotropic particles (e.g. such as shown in FIG. 1(d)), is typically brought into contact with topical surface of a body (e.g. skin) and the customized anisotropic particles are embedded into said topical surface using an externally applied force by an embedding action that is at least one of a scrubbing action, a rubbing action, a flow, a shear flow, and extensional flow, a pressing, a compressing, a scraping, and a spraying, according to an embodiment of the current invention. According to an embodiment of the current invention, a polymer material (lines) and a fluid material (light shaded region) typically remain above the skin subsequent to the embedding of the custom-shaped particles into the skin by an embedding action. According to another embodiment of the current invention, some customized anisotropic particles remain above the skin surface, although it is typically desirable for most or nearly all of the custom-shaped and custom-sized particles to be embedded into the skin by an embedding action.

FIG. 1(f) is a schematic illustration of a plurality of customized anisotropic particles (dark triangular prisms) composed of a particle material that is suitable for a topical scrub, wherein said particles have been embedded into a topical surface such as skin (i.e. at least partially into or below the surface of the skin) after an embedding action, according to an embodiment of the current invention. According to an embodiment of the current invention, typically the shape and size of the particles is chosen to facilitate embedding from a scrubbing motion of the dispersion of particles in a gel-like fluid material; a pointed shape feature, such as a point on a triangular prism, can facilitate such embedding. According to another embodiment of the current invention, a gel-like fluid material containing a polymer material (and possibly also unembedded particles) can be washed away from the skin after an embedding action, while still leaving a plurality of particles embedded below the skin.

FIG. 1(g) is a schematic illustration of the dissolution over time of a plurality of customized anisotropic particles composed of a particle material that is suitable for a topical scrub, wherein said customized anisotropic particles have been embedded into skin subsequent to an embedding action, according to an embodiment of the current invention. According to an embodiment of the current invention, as said particle material is removed by natural biological processes while said particles remain embedded into skin, any component material within said particle material is released into neighboring tissue surrounding the particle, thereby providing a therapeutic effect to said neighboring tissue that is potentially localized. According to an embodiment of the current invention, said particle material is typically a combination of a solid bio-compatible carrier material (e.g. a collagen or an albumin) and a bio-active loading material (e.g. a drug such as anti-fungal drug molecules). According to an embodiment of the current invention, said customized anisotropic particles are designed to have a customized shape, a customized size, and a customized composition of a carrier material and a loading material that not only facilitates embedding, but also provides a desired time-release profile for a bio-active loading material.

FIG. 2 is an optical micrograph image of a plurality of customized anisotropic particles that have been embedded into a topical surface of an epidermal layer of human skin by an embedding action, according to an embodiment of the current invention. The customized anisotropic particles are monodisperse square-cross platelets (shown as small darker cross-like regions having a distribution of positions and orientations); each customized anisotropic particle has a square cross-shaped cross-section with four arms, has been produced using a method of spatially patterned radiation by ultraviolet stepper lithography, has a maximum spatial dimension of 4.5 microns, and is 1 micron thick. The particle material is a photo-crosslinkable photoresist (AZ5214), which has been selected for the purposes of demonstration because its relatively strong optical absorption property, compared to that of the skin, provides adequate contrast for the purposes of showing by optical microscopy that the particles have been embedded into skin by an embedding action. Some particles embedded into the skin are not in focus, so not all embedded particles present are actually visible in the micrograph.

FIG. 3 is a schematic illustration of a system for producing a topical scrub according to an embodiment of the current invention.

### DETAILED DESCRIPTION

In describing embodiments of the present invention illustrated in the drawings, specific terminology is employed for the sake of clarity. However, the invention is not intended to be limited to the specific terminology so selected. It is to be understood that each specific element includes all technical equivalents which operate in a similar manner to accomplish a similar purpose.

According to an embodiment of the current invention, a material suitable for a topical scrub is typically composed of at least a fluid material in which one or more customized anisotropic particles having customized shape, customized size, and customized composition are dispersed. According to an embodiment of the current invention, a customized anisotropic particle, which has a customized shape, customized size, and customized composition, is fabricated using at least one of a lithographic method, an optical lithographic method, a patterning method, an etching method, an irradiation method, an imprinting method, an extrusion method, a templating method, a stamping method, a nanoimprinting method, a direct-write method, a deposition templating method, a relief deposition templating method, a relief radiation templating method, a patterned plate imprinting method, a bottom-up particle synthesis reaction in solution, a crystal growth method, an erosion method, and a deformation method.

According to an embodiment of the current invention, the fluid material typically contains at least one of a liquid (e.g. water or oil), a gas, a liquid crystal, a viscoelastic material, a yield-stress material, a viscoplastic material, a foam, an emulsion, a nanoemulsion, a polymer solution, a polypeptide solution, a co-polypeptide solution, and a gel-like material. According to an embodiment of the invention, the fluid material, if placed in contact with a patch of skin, typically possesses rheological properties that enable it to remain largely on the skin in the place where it was contacted to the skin without significant flowing due to gravitational forces that could cause the fluid material to flow to regions of the skin other than the point of original contact over time scales of several minutes

According to an embodiment of the current invention, a customized anisotropic particle suitable for a topical scrub can be made by a variety of processes, including but not limited to top-down lithographic methods (e.g. such as at least one of relief deposition templating and spatially patterned radiation) and also bottom-up synthesis (e.g. nucleation and growth of particles in a solution). According to an embodiment of the current invention, a customized anisotropic particle suitable for a topical scrub typically has a maximum spatial dimension that is near or below one millimeter. In some embodiments of the current invention, a customized anisotropic particle suitable for a topical scrub is several millimeters in its longest spatial dimension, but sub-millimeter in its other spatial dimensions. According to other embodiments of the current invention, a maximum volume of a customized anisotropic particle suitable for a topical scrub is typically near or below about one cubic millimeter. According to an embodiment of the current invention, a maximum dimension of a customized anisotropic particle suitable for a topical scrub is less than about one millimeter. According to an embodiment of the current invention, a maximum dimension of a customized anisotropic particle suitable for a topical scrub is typically less than about one hundred microns.

According to an embodiment of the current invention, it is typically preferable to optimize the shape and size of a customized anisotropic particle by designing at least some aspects of the shape of said customized anisotropic particle such that said customized anisotropic particle can easily embed into a soft topical surface through an embedding action, such as scrubbing, that applies forces to said customized anisotropic particle. According to an embodiment of the current invention, a desirable shape feature of a customized anisotropic particle is at least one of a point, a corner, a projection, a barb, a spike, an edge, a sharp tip, a sharp edge, a protuberance, a hook, and a wedge. According to an embodiment of the current invention, a desirable shape feature of a customized anisotropic particle is designed and imparted to the particle by means of at least one of spatially patterned radiation, relief deposition templating, relief radiation templating, stamping, spatially patterned deposition, lithography, etching, development, and imprinting.

According to another embodiment of the current invention, a shape feature in a customized anisotropic particle is designed to obtain a desired time-release profile of a material constituent within said customized anisotropic particle subsequent to embedding said customized anisotropic particle into a selected topical surface. According to some embodiments of the current invention, one or more shape features of a customized anisotropic particle are designed to provide a combination of a facilitating of embedding of said customized anisotropic particle into a selected topical surface and a releasing of a material constituent within said customized anisotropic particle according to a desired time-release profile into said topical surface where said customized anisotropic particles have been embedded.

According to an embodiment of the current invention, a customized anisotropic particle is composed of a particle material that contains at least one of a biologically derived material, a bio-compatible material, a bio-inert material, a bio-degradable material, a bio-transportable material, a bio-active material, a bio-therapeutic material, a bio-protective material, a bio-toxic material, a bio-nontoxic material, a bio-stabilizing material, a bio-absorbable material, a bio-enhancing material, a bio-dispersible material, a bio-marking material, a bio-nutrient material, a bio-replenishing material, a bio-soothing material, a bio-growth-enhancing material, a bio-growth-inhibiting material, a bio-stimulant material, an anti-inflammatory material, an anti-microbial material, an anti-fungal material, an anti-cancer material, an anti-(hair-loss) material, an anti-itch material, an anti-insect material, an anti-arachnid material, an antiseptic material, an anti-wart material, an anti-(skin mole) material, an anti-(skin blemish) material, an anti-(skin birth mark) material, an anti-pain material, an anesthetic material, an anti-arthritis material, a (hair growth)-stimulant material, a (hair growth)-promoting material, a bio-blocking material, a pore-opening material, an anti-acne material, an anti-wrinkle material, an anti-burn material, an anti-(skin burn) material, a hygroscopic material, a depilatory material, an anti-shingle material, an anti-(cold sore) material, an anti-bursitis material, an anti-callous material, an anti-(foot corn) material, an anti-ulcer material, an anti-leprosy material, an anti-(sexually transmitted disease) material, an anti-viral material, an anti-(poison ivy) material, an anti-(poison oak) material, and anti-(poison sumac) material, a bio-scaffold material, a polypeptide material, a copolypeptide material, a lipophilic material, an amphiphilic material, a hydrophilic material, a hydrophobic material, a lipoprotein material, a genetic material, an enzymatic material, a bio-catalytic material, a bio-inhibitory material, a steroid material, an anti-odor material, an anti-irritation material, a fragrance, an essential oil material, an isotopic material, a radio-isotopic material, a magnetically-responsive material, an electrically responsive material, an electromagnetically-responsive material, an optically responsive material, a sun-blocking material, a sun-screening material, a fluorescent material, an optically absorbing material, an optically scattering material, an imaging contrast-enhancing material, an organic material, an inorganic material, a metallic material, a semiconducting material, an organometallic material, a nanoporous material, a microporous material, a polymeric material, a biopolymeric material, a waxy material, an embalming material, a preservative material, a photoresist material, a photo-crosslinkable material, crosslinkable material, a biopolymeric crosslinkable material, an analgesic material, an acidic material, a basic material, a neutral material, a thermally reactive material, and a thermally-responsive material.

According to some embodiments of the current invention, a customized anisotropic particle is composed of a particle material that contains at least one of an anti-(bacterial infection) material, an anti-pyoderma material, an anti-impetigo material, an anti-(pityriasis alba) material, an anti-(streptococcal intertrigo) material, an anti-(otitis externa) material, an anti-folliculitis material, an anti-furunculosis material, an anti-(methicillin resistant staph. aureus) material, an anti-(staph. aureus) material, an anti-(necrotising fasciitis) material, an anti-(scarlet fever) material, an anti-erythrasma material, an anti-(pitted keratolysis) material, an anti-(trichomycosis axillaris) material, an anti-gonorrhoea material, an anti-(meningococcal disease) material, an anti-erysipeloid material, an anti-chancroid material, an anti-(athlete's foot) material, an anti-(jock itch) material, an anti-(gram negative folliculitis) material, an anti-paronychia material, an anti-(spa pool folliculitis) material, an anti-(granuloma inguinale) material, an anti-anthrax material, an anti-syphilis material, an anti-(Lyme disease) material, an anti-(cat scratch fever) material, an anti-(bacillary angiomatosis) material, an anti-(Kawasaki disease) material, an anti-(pseudofolliculitis barbae) material, an anti-sarcoidosis material, an anti-(skin infection) material, an anti-(erythematous rash) material, an anti-(nappy rash) material, an anti-(diaper rash) material, an anti-intertrigo material, an anti-(fungal infection) material, an anti-ringworm material, an anti-favus material, an anti-thrush material, an anti-(tinea capitis) material, an anti-(tinea corporis) material, an anti-(tinea cruris) material, an anti-(tinea pedis) material, an anti-onychomycosis material, an anti-(virus infection) material, an anti-(herpes simplex) material, an anti-(herpes zoster) material, an anti-(molluscum contagiosum) material, an anti-(Kaposi sarcoma) material, an anti-(plantar warts) material, an anti-(condyloma acuminatum) material, an anti-ecthymacontagiosum material, an anti-(skin tumor) material, an anti-tumor material, an anti-fibroma material, an anti-(knuckle pad) material, an anti-(cutaneous tag) material, an anti-keloid material, an anti-lipoma material, an anti-leiomyoma material, an anti-neuroma material, an anti-(Glomus tumor) material, an anti-(seborrhoeic warts) material, an anti-keratoses material, an anti-(basal cell epithelioma) material, an anti-(squamous cell epithelioma) material, an anti-(intra-epidermal epithelioma) material, an anti-(Paget's disease) material, an anti-(malignant melanoma) material, an anti-(mycosis fungoides) material, an anti-(Hodgkin's disease) material, an anti-(dermoid cyst) material, an anti-(pilonidal cyst) material, an anti-(benign cystic epithelioma) material, an anti-syringoma material, an anti-(sebaceous cyst) material, an anti-milium material, an anti-(mucous cyst) material, an anti-hydrocystoma material, an anti-(tinea versicolor) material, an anti-candidiasis material, an anti-biotic material, and an anti-erysipelas material.

According to other embodiments of the current invention, a customized anisotropic particle is composed of a particle material that contains at least one of an anti-arthritis material, an anti-gangrene material, an anti-eczema material, an anti-dandruff material, an anti-tattoo material, an anti-(skin ulcer) material, an anti-(skin lesion) material, an anti-(skin boil) material, an anti-psoriasis material, an anti-dermatitis material, an anti-(skin rash) material, an anti-necrosis material, an anti-(frost bite) material, an anti-(varicose vein) material, an anti-(spider vein) material, an anti-cellulite material, an anti-(insect bite) material, an anti-(spider bite) material, an anti-(tick bite) material, an anti-scar material, an anti-chafing material, an anti-sweat material, a deodorant material, and an anti-hives material.

According to an embodiment of the current invention, a customized anisotropic particle is composed of a particle material that contains at least one of an antimycotic drug molecule, a polyene antimycotic drug molecule, an imidazole drug molecule, a triazole drug molecule, a thiazole drug molecule, an allylamine drug molecule, an enchinocandins drug molecule, a ciclopirox drug molecule, a tolnaftate drug molecule, and an allicin drug molecule.

According to an embodiment of the current invention, a customized anisotropic particle is composed of a particle material that contains at least one of an antibiotic drug molecule, a beta-lactam drug molecule, a tetracycline drug molecule, an aminoglycoside drug molecule, a rifamycin drug molecule, a quinolone drug molecule, a macrolide drug molecule, and a sulphonamide drug molecule.

According to an embodiment of the current invention, a customized anisotropic particle is composed of a particle material that contains at least one of an anti-inflammatory drug molecule, a salicylate drug molecule, a steroidal drug molecule, a corticosteroidal drug molecule, a non-steroidal anti-inflammatory drug molecule, a proprionic acid drug molecule, an acetic acid drug molecule, an enolic acid drug molecule, a fenamic acid drug molecule, a selective COX-2 inhibitor drug molecule, an ibuprofen drug molecule, a naproxen drug molecule, a dicolfenac drug molecule, a hydroxychloroquine drug molecule, a penicillamine drug molecule, an azathioprine drug molecule, a sulfasalazine drug molecule, and a methotrexate drug molecule.

According to an embodiment of the current invention, a customized anisotropic particle is composed of a particle material that contains a hair re-growth drug molecule. According to another embodiment of the current invention, a customized anisotropic particle is composed of a particle material that contains a minoxidil drug molecule suitable for re-growing hair.

According to an embodiment of the current invention, a customized anisotropic particle is composed of a particle material that contains a trichloroacetic acid molecule suitable for tattoo removal.

According to an embodiment of the current invention, a shear elastic modulus that characterizes a particle material of a customized anisotropic particle, which is dispersed in a fluid material, is typically at least comparable to or greater than the shear elastic modulus of a topical surface into which said customized anisotropic particle is to be embedded. According to another embodiment of the current invention, it is typically preferable to ensure that a yield stress that characterizes a particle material of a customized anisotropic particle dispersed in a fluid material is significantly greater than a yield stress of a topical surface into which said customized anisotropic particle is to be embedded. According to an embodiment of the current invention, a particle material of said customized anisotropic particle typically has greater elastic strength than a biological tissue at and immediately below a selected topical surface (e.g. a portion of skin on a body) in order to facilitate embedding of said customized anisotropic particle into said selected topical surface.

According to an embodiment of the current invention, a customized anisotropic particle is composed of a particle material that typically does not dissolve or otherwise degrade substantially while in a fluid material. According to some embodiments of the current invention, at least one of a particle material and a fluid material are selected so that the particle material is at least partially insoluble in the fluid material. According to an embodiment of the current invention, a fluid material is chosen to provide a long storage life of a customized anisotropic particle composed of a particle material in said fluid material. According to another embodiment of the current invention, a fluid material is selected to confer not only a long storage life of a customized anisotropic particle in said fluid material, but is also selected to be bio-compatible (e.g. non-irritating) with a selected topical surface of a body.

According to an embodiment of the current invention, subsequent to contacting a topical scrub containing customized anisotropic particles to skin and subsequent to applying an embedding action (e.g. a scrubbing action), one or more customized anisotropic particles become embedded in at least one of a layer of an epidermis, a layer of a dermis, a layer of a sub-cutaneous tissue, a stratum corneum, a stratum lucidum, a stratum granulosum, a stratum germinativum, a dermal papillus, a papillary dermis, a reticular dermis, an ocular membrane, a nasal membrane, an oral membrane, an aural membrane, a vaginal membrane, a labial tissue, an intestinal membrane, an anal membrane, a urethral membrane, and a mucosal membrane.

According to an embodiment of the current invention, a rheological modifier material is added to a fluid material in which customized anisotropic particles have been dispersed to create a topical scrub material having a gel-like rheological property. According to an embodiment of the current invention, a rheological modifier material is added to a fluid material in which customized anisotropic particles have been dispersed to create a topical scrub material having a yield stress in excess of 10 Pa, and more desirably in excess of 100 Pa. According to an embodiment of the current invention, a rheological modifier material added to a fluid material in which customized anisotropic particles have been dispersed is at least one of a polymer, a copolymer, a biopolymer, a diblock co-polymer, a star polymer, a high molecular weight polymer, a surfactant, a lipid, a lipoprotein, an amphiphilic material, a hydrogel, a polypeptide, a co-polypeptide, a dendrimer, a starch, a modified starch, an emulsion, a nanoemulsion, a nanoparticulate dispersion, a cream, a foam, a jelly, a lyotropic liquid crystal, a microgel particle, a micellar solution, a gel, a thermo-responsive gelling material, a thermo-reversible gelling material, a crosslinker, and a crosslinking agent.

According to an embodiment of the current invention, a material produced is a topical scrub that is effective after a single-application to a topical surface, contains penetrating customized anisotropic particles, is therapeutic, and has gel-like rheological properties. According to another embodiment of the current invention, said topical scrub contains biodegradable custom-shaped particles that provide long-time release of drug molecules into at least one of a layer of the epidermis, a layer of the dermis, a layer of sub-cutaneous tissue, and a sub-dermal layer.

According to an embodiment of the current invention, customized anisotropic particles, which have penetrating and therapeutic properties, are produced by a process involving: selecting a particle material that is biodegradable and biocompatible (e.g. biopolymers such as poly-L-lactic acid, collagen, and/or polypeptides); selecting therapeutic drug molecules that provide a desired therapy

(e.g. anti-fungal drugs such as -conazoles and/or anti-inflammatory drugs); incorporating said therapeutic drug molecules into said particle material at a desired dose (e.g. via solution processing of particle material plus drug molecules); designing a size and shape (e.g. sub-100 micron and/or colloidal particles that have a pointed shape) of customized anisotropic particles to facilitate penetration of particles into a topical surface during and embedding action, such as rubbing; mass-producing therapeutic customized anisotropic particles (e.g. using methods such as relief deposition templating); making a liquid-based gel containing therapeutic customized anisotropic particles (e.g. by adding a biocompatible polymer that forms a viscoelastic gel to a dispersion of custom-shaped particles containing drugs molecules); and applying said liquid-based gel containing therapeutic customized anisotropic particles to a topical surface of a body by contacting with said topical surface and generating an embedding action (e.g. by rubbing, shearing, massaging, and/or compressing of said liquid-based gel containing therapeutic customized anisotropic particles against or into said topical surface), causing at least a portion of said therapeutic customized anisotropic particles to embed into said topical surface.

According to an embodiment of the current invention, therapeutic customized anisotropic particles in a gel-like topical scrub can be embedded by a single application by at least one of a patient (e.g. who has a skin ailment), doctor, and nurse, wherein said therapeutic customized anisotropic particles offer a controllable long time release, that provides the following advantages: there is no need to reapply medication to an affected area topically every 12 to 24 hours for 1 to 2 weeks; a means of localized one-time treatment; an efficient use of drug molecules; and said topical surface (e.g. skin) can be rinsed after a single application of gel-like topical scrub without significantly affecting therapy provided by said therapeutic customized anisotropic particles that have been embedded into said affected area of said topical surface. According to an embodiment of the current invention, said gel-like topical scrub has over-the-counter uses for anti-fungal therapies. According to an embodiment of the current invention, said gel-like topical scrub has over-the-counter uses for anti-inflammation therapies. [ 0045] According to an embodiment of the current invention, customized anisotropic particles that have pointed features penetrate and remain embedded into and below a topical surface (e.g. skin) significantly more easily than spherical particles subsequent to a rubbing action. According to another embodiment of the current invention, a patient having a region of a topical surface affected by a malady wears a glove to protect a hand and applies a topical scrub containing customized anisotropic particles by contacting the topical scrub with the affected region of the topical surface and scrubbing said topical scrub into the affected area using a protected hand with a motion that applies pressure and/or shear to the topical scrub that causes at least a portion of said customized anisotropic particles to embed into an affected region of a topical surface for the purposes of treating said malady said affected region.

According to an embodiment of the current invention, a range of sizes, shapes, and concentrations of active drug molecules in therapeutic customized anisotropic particles contained within a topical scrub can be used to control the time-release profile of said active drug molecules after application of that topical scrub to a topical surface caused by an embedding action.

According to an embodiment of the current invention, a topical scrub containing therapeutic customized anisotropic particles provides efficient localized treatment of at least one of a dermal malady and a sub-dermal malady using particles that have custom-designed shapes that facilitate embedding and provide a long time release profile for a single-application treatment.

According to an embodiment of the current invention, scaling-up production of therapeutic customized anisotropic particles for use in a topical scrub is accomplished by replicating templates used in relief deposition templating, so remote production facilities can fabricate therapeutic customized anisotropic particles without expensive lithographic exposure technology.

According to an embodiment of the current invention, a composition of a particle material suitable for deposition by a liquid coating method (e.g. by wiping, spray coating, or spin-coating) onto a pillar template for the purposes of pillar deposition templating is typically made as follows: a first solution of a carrier material (e.g. such as a solution of a collagen, an albumin, and/or a poly-lactic acid) in a first solvent is prepared at a pre-selected concentration of carrier material; a second solution of a loaded material (e.g. such as therapeutic drug molecules) is prepared at a pre-selected concentration of loading material in a second solvent that is at least partially miscible with the first solvent; and said first uniform solution of a carrier material is mixed together with said second uniform solution of a loaded material in desired proportions to provide a desired ratio of mass of loaded material relative to mass of carrier material.

According to an embodiment of the current invention, a carrier material for customized anisotropic particles in a topical scrub is typically composed of at least one of a protein, an enzyme, an acid, a base, a vitamin, a nutrient, a collagen, a gelatin, an albumin, an actin, a tubulin, a polypeptide, a copolypeptide, a polysaccharide, a modified polysaccharide, a poly-lactic acid, a cellulose, a starch, a modified starch, a polymer, and a biopolymer.

According to an embodiment of the current invention, a particle volume fraction of customized anisotropic particles in a topical scrub is typically between 0.01% and 30%, where said particle volume fraction is defined as the volume of said customized anisotropic particles present within a given volume of said topical scrub divided by said given volume of said topical scrub.

According to an embodiment of the current invention, subsequent to said embedding action, a cleansing action is applied to remove unembedded material (e.g. gel-like fluid material) above a topical surface without causing detachment of at least a majority of customized anisotropic particles that have been embedded into said topical surface by said embedding action. According to another embodiment of the current invention, a shape feature of a customized anisotropic particle is designed to inhibit detachment of and therefore enhance retention of a customized anisotropic particle that has been embedded into a topical surface by an embedding action. According to an embodiment of the current invention, a shape feature that enhances retention of a customized anisotropic particle that has been embedded into a topical surface subsequent to an embedding action is at least one of a barb, a hook, a flange, a notch, a serration, an arm, a spoke, and a roughened surface.

According to an embodiment of the current invention, a dispersion of customized anisotropic particles suitable for a topical scrub contains customized anisotropic particles wherein each particle has substantially the same shape and substantially the same volume, corresponding to a highly uniform shape distribution and highly uniform volume distribution. A dispersion of customized anisotropic particles having a highly uniform shape distribution and a highly uniform size distribution can be obtained using a variety of particle production methods, including but not limited to relief deposition templating, relief radiation templating, and spatially patterned radiation. A dispersion of customized anisotropic particles having a highly uniform shape distribution and highly uniform volume distribution can be advantageous in providing a predetermined time-release profile of a particle material in a topical surface after an embedding action that embeds a plurality of customized anisotropic particles into said topical surface.

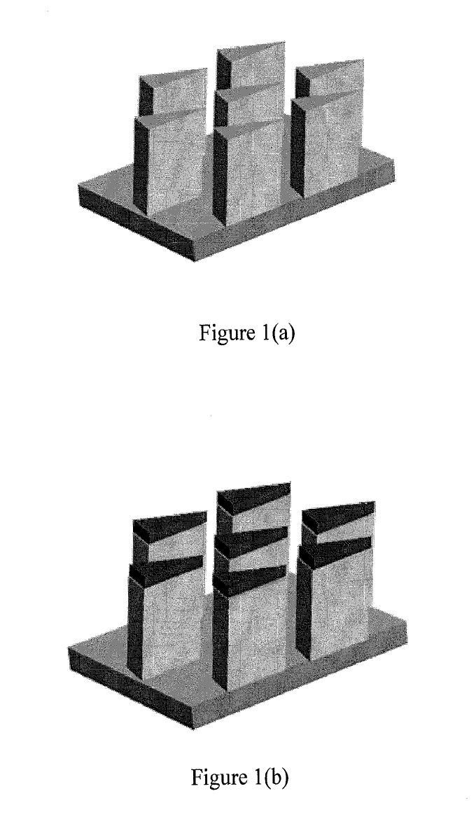

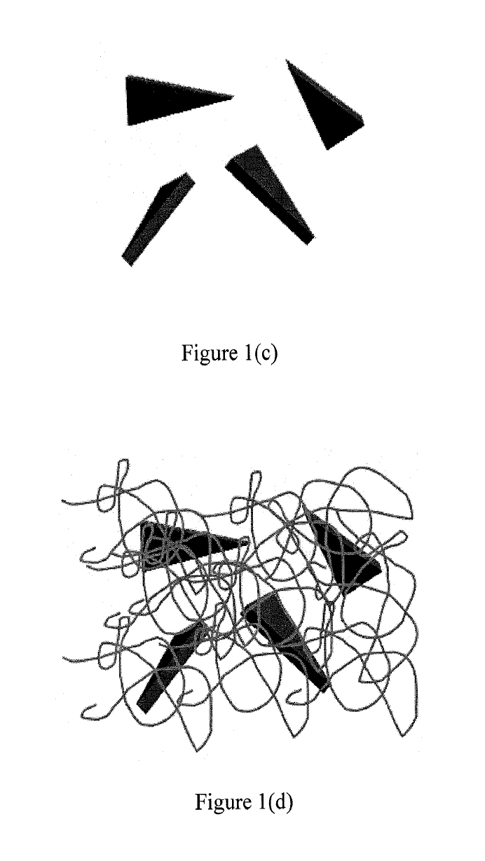

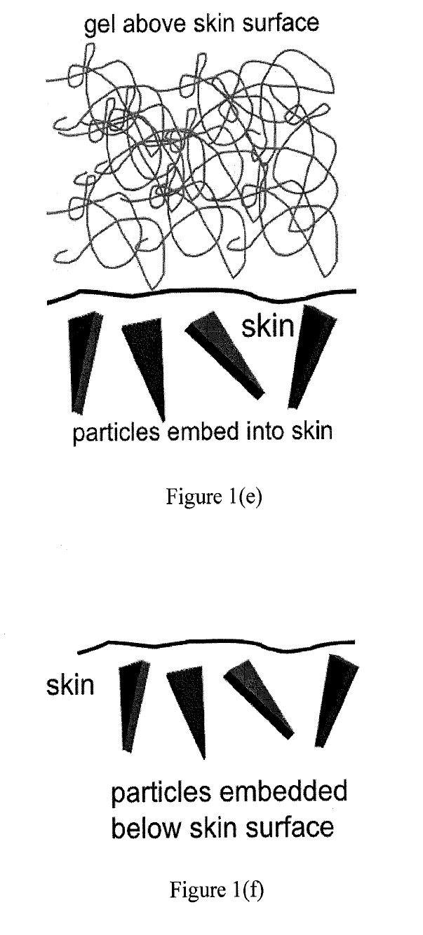

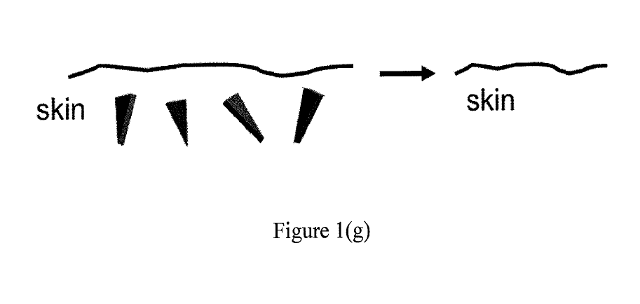

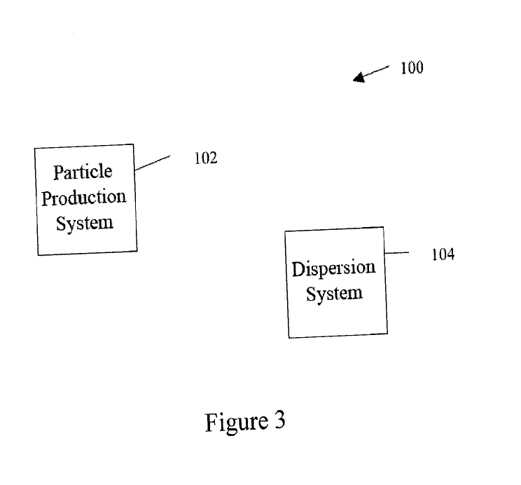

FIG. 3 is a schematic illustration of a system for producing a topical scrub 100 according to another embodiment of the current invention. The system 100 has a particle production system 102 constructed to produce a plurality of customized anisotropic particles and a dispersion system 104 arranged proximate the particle production system to be able to receive a plurality of customized anisotropic particles from the particle production system. The particle production system 102 can be a system for producing particles using relief deposition templating, a system for producing particles using spatially patterned radiation, or a system for producing particles using an imprinting method, for example. For example, the particle production system 102 can include a pillar template such as that of the example illustrated schematically in FIG. 1(a). The dispersion system 104 can be a fluidic mixing system, a millifluidic mixing system, or a microfluidic mixing system, for example. The dispersion system 104 may contain and/or receive a fluid material in which the customized anisotropic particles can be dispersed. The fluid material may contain a rheological modifier and/or a stabilizing agent to inhibit particle aggregation. In operation for example, the system 100 can contain a particle production system 102 based on pillar deposition templating, and a solution of a particle material containing a biocompatible biopolymeric as a carrier material and drug molecules as a loading material is deposited by contacting the pillar template with a liquid interface of said solution of a particle material, and the particle material is solidified on the pillars by evaporative heating of said solution to form a plurality of customized anisotropic particles. Said plurality of customized particles formed on the pillars of the pillar template are transferred to the dispersion system 104, wherein the particles are released from the template and dispersed into a fluid material by fluid flow and agitation generated by a flow-producing device, such as a mixer or pump. Typically, the pillar template is re-used by the particle production system to produce additional customized anisotropic particles.

### A First Example Embodiment

The following example embodiment describes a process for fabricating a topical scrub containing one or more customized anisotropic particles and applying said topical scrub to a selected topical surface to effectively treat said selected topical surface.

Customized anisotropic particles are mass-produced for a topical scrub using pillar deposition templating, a form of relief deposition templating. A portion of a solid pillar template is created (e.g. using a material such as silicon) as shown in FIG. 1(a), typically by a lithographic process. The triangular cross-sectional shapes of the pillars on the template have been designed to impart pointed shape features to the customized anisotropic particles. A pillar template typically has a spatial dimension of about 100 mm or larger, and a pillar on the template typically has a maximum cross-sectional dimension that is less than about one millimeter. Optionally, the surfaces of a pillar template can be coated with a release agent. Onto at least the top surfaces of the pillars on the pillar template, a particle material containing therapeutic drug molecules is deposited (e.g. using a coating process such as spin-coating or spray-coating) and solidified (e.g. using heating or evaporation) to form a plurality of customized anisotropic particles containing therapeutic drug molecules, as shown in FIG. 1(b). Subsequently, customized anisotropic particles containing therapeutic drug molecules are released from the pillar template into a fluid material, such as a simple viscous liquid, as shown in FIG. 1(c). Since customized anisotropic particles can be produced in a manner that does not alter the pillar template, the pillar template can be repeatedly re-used to make additional customized anisotropic particles. In order to provide desired gel-like rheological properties to the topical scrub, a high molecular weight polymer material is added in sufficient quantity and mixed with the simple viscous liquid containing customized anisotropic particles, as shown in FIG. 1(d), yielding a topical scrub that is a liquid gel-like material within which customized anisotropic particles containing drug molecules are dispersed.

Given a gel-like topical scrub containing therapeutic customized anisotropic particles, such as has been produced through FIGS. 1(a)-1(d), the gel-like topical scrub is applied to an affected area of a topical surface (e.g. an area of skin affected by a condition or malady) as follows. A quantity of the topical scrub (e.g. typically at least ten microliters) is brought into contact with the affected area. A scrubbing motion is applied to the topical scrub on the affected area (e.g. using a finger or hand protected by a glove), and this embedding action causes customized anisotropic particles to embed into the affected area of the topical surface, such as skin, as shown in FIG. 1(e). In some cases, an embedding action is generated by a patient who has an affected area of a topical surface (e.g. providing a form of self-treatment); the patient scrubs the gel-like topical scrub onto a local affected area of skin using a glove that prevents transfer of particles into skin of the hand that is applying the scrub. In some instances, the scrubbing motion is applied for one or more minutes to achieve a desired surface concentration of embedded customized anisotropic particles. Subsequent to the embedding action, any residual gel-like material and un-embedded particles above the topical surface are typically rinsed off without significantly affecting the embedded particles, as shown in FIG. 1(f).

Subsequently, as embedded customized anisotropic particles biodegrade and/or dissolve, drug molecules are released over a controllable long time period, as shown in FIG. 1(g); the biodegradation and dissolution of particle material releases drug molecules locally to the vicinity of the affected area of the topical surface. The released drug molecules provide a desired therapy at and below the topical surface, and the size and shape of the customized anisotropic particles provides a controllable time-release profile of the drug molecules to the affected area of the topical surface.

### A Second Example Embodiment

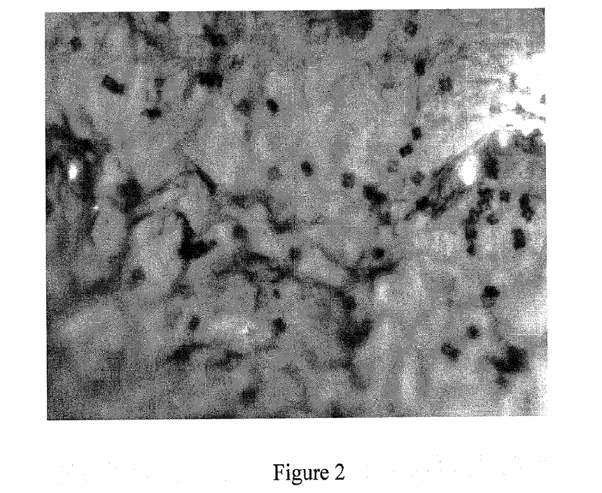

An example of a plurality of customized anisotropic particles embedded into an epidermal layer of human skin after an embedding action is shown in the optical microscope image in FIG. 2. Said customized anisotropic particles (see the plurality of dark cross-shaped regions at different positions and orientations) are sub-millimeter, plate-like, have a cross section in the shape of a square cross, a maximum spatial dimension of 4.5 microns and a thickness of 1 micron, are composed of a photo-crosslinkable polymeric material (photoresist AZ5214), and have been created using a method of spatially patterned radiation by ultraviolet stepper lithography. These cross-shaped anisotropic particles have been dispersed into water at a particle volume fraction of about 0.3%, contacted with an epidermal layer of human skin, and a scrubbing action (rotary motion with a glove-protected finger while applying pressure) has been applied for thirty seconds, causing the cross-shaped particles to embed into the skin. The four arms on the cross-shaped particles facilitate retention of the particles in the skin subsequent to the embedding action.

We claim:

1. A method of producing a topical scrub, comprising:
providing a particle material suitable for producing a customized anisotropic particle;
producing a plurality of customized anisotropic particles composed of said particle material, each customized anisotropic particle having a particle shape, a particle volume, and a particle composition; and
dispersing said plurality of customized anisotropic particles in a fluid material to form a dispersion of customized anisotropic particles in said fluid material, wherein at least a feature of said particle shape facilitates embedding at least a portion of said plurality of customized anisotropic particles into a topical surface by a scrubbing action.

2. A method of producing a topical scrub according to claim 1, wherein said producing a plurality of customized anisotropic particles utilizes at least one of a lithographic method, a templating method, an imprinting method, a stamping method, an extrusion method, a relief deposition templating method, a pillar deposition templating method, a well deposition templating method, a spatially patterned radiation exposure method, an etching method, a relief radiation templating method, a particle templating method, a two-patterned surface imprinting method, a bottom-up solution synthesis method, and a patterned deposition method.

3. A method of producing a topical scrub according to claim 1, further comprising modifying a rheological property of said dispersion of customized anisotropic particles by adding a rheological modifier to increase at least one of a viscosity, a resistance to stress, an elastic shear modulus, a yield stress, and a magnitude of a viscoelastic complex shear modulus of said dispersion of customized anisotropic particles in said fluid material.

4. A method of producing a topical scrub according to claim 3, wherein said rheological modifier is at least one of a polymer, a biopolymer, a copolymer, a diblock co-polymer, a star polymer, a high molecular weight polymer, a surfactant, an amphiphilic material, a hydrogel, a polypeptide, a co-polypeptide, a dendrimer, a starch, a modified starch, an emulsion, a nanoemulsion, a nanoparticulate dispersion, a cream, a foam, a jelly, a lyotropic liquid crystal, a microgel particle, a micellar solution, a thickener, a gel, a thermo-responsive gelling material, a thermo-reversible gelling material, a crosslinker, and a crosslinking agent.

5. A method of producing a topical scrub according to claim 1, wherein said particle volume of each of said plurality of customized anisotropic particles is less than about one cubic millimeter.

6. A method of producing a topical scrub according to claim 1, wherein a maximum spatial dimension of each of said plurality of customized anisotropic particles is less than about one millimeter.

7. A method of producing a topical scrub according to claim 1, wherein said particle material contains at least one of a carrier material and a loading material.

8. A method of producing a topical scrub according to claim 7, wherein said carrier material is at least one of a bio-compatible material, a bio-degradable material, a bio-inert material, a bio-polymer material, a bio-erodable material, a bio-nutrient material, a bio-inhibitor material, a bio-catalytic material, a bio-reactive material, a bio-digestible material, a bio-absorbable material, a bio-derived material, a protein, an enzyme, a vitamin, a nutrient, a collagen, a gelatin, an albumin, an actin, a tubulin, a polypeptide, a copolypeptide, a polysaccharide, a starch, a modified starch, a porous material, an elastic material, and a biopolymer.

9. A method of producing a topical scrub according to claim 7, wherein said loading material is at least one of a therapeutic material, an anti-fungal material, an anti-viral material, an anti-bacterial material, an anti-microbial material, an anti-wart material, an anti-blemish material, an anti-cancer material, an anti-irritation material, an anti-scar material, an anti-inflammatory material, an anesthetic material, an anti-septic material, and an anti-burn material.

10. A method of producing a topical scrub according to claim 1, wherein said at least a feature of said particle shape is at least one of a point, a corner, an edge, a barb, a spike, a protuberance, a blade, a sliver, an acute angle, a hook, and a needle.

11. A method of producing a topical scrub according to claim 7, wherein at least one of said particle shape, said particle volume, and said particle composition is designed to provide a predetermined time-release profile of said loading material subsequent to said embedding.

12. A method of producing a topical scrub according to claim 1, wherein a volume fraction of said customized anisotropic particles in said dispersion of anisotropic particles is between about 0.01% and about 30%.

13. A method of producing a topical scrub according to claim 1, wherein said plurality of customized anisotropic particles in said fluid material remain stable against at least one of separation, aggregation, agglomeration, and degradation prior to said embedding.

14. A method of producing a topical scrub according to claim 1, wherein said embedding causes one or more customized anisotropic particles to substantially penetrate and remain within at least one of a layer of an epidermis, a layer of a dermis, a layer of a sub-cutaneous tissue, a stratum corneum, a stratum lucidum, a stratum granulosum, a stratum germinativum, a dermal papillus, a papillary dermis, a reticular dermis, a follicle, a pore, an ocular membrane, a nasal membrane, an oral membrane, an aural membrane, a vaginal membrane, a labial tissue, an intestinal membrane, an anal membrane, a urethral membrane, and a mucosal membrane.

15. A method of producing a topical scrub according to claim 11, wherein said time-release profile provides a predetermined release of said loading material over at least a duration of three days.

16. A multi-component composition, comprising:
a first material component in which customized anisotropic particles can be dispersed; and
a plurality of customized anisotropic particles dispersed in said first material component, each customized anisotropic particle having a particle shape, a particle volume, and a particle composition,
wherein at least a feature of said particle shape facilitates embedding at least a portion of said plurality of customized anisotropic particles into a topical surface by a scrubbing action, and
wherein said plurality of customized anisotropic particles is at least 100 particles.

17. A multi-component composition according to claim 16, wherein said first material component is at least one of a fluid, a liquid, a polymeric solution, a biopolymer solution, a dispersion, a mixture, a gel, an emulsion, a nanoemulsion, or a solution, such that said multi-component composition is a topical scrub.

18. A multi-component composition according to claim 16, wherein said customized anisotropic particles contain at least one of a therapeutic material, an anti-fungal material, an anti-viral material, an anti-bacterial material, an anti-microbial material, an anti-wart material, an anti-blemish material, an anti-cancer material, and an anti-burn material, such that said multi-component material is a topical scrub.

19. A system for producing a topical scrub, comprising:
a particle production system structured to produce a plurality of customized anisotropic particles; and
a dispersion system arranged proximate said particle production system to be able to receive a plurality of said customized anisotropic particles and to be able to receive a fluid material, and through which said plurality of customized anisotropic particles can be dispersed in said fluid material,
wherein each of said plurality of customized anisotropic particles has at least a feature of a shape that facilitates embedding of at least a portion of said customized anisotropic particles into a topical surface by a scrubbing action.
